(19) United States
(12) Patent Application Publication
(10) Pub. No.: US 2012/0251615 A1
(43) Pub. Date: Oct. 4, 2012

(54) AGENT FOR INTRA-ARTICULAR INJECTION

(76) Inventor: Horst KIEF, Ludwigshafen (DE)

(21) Appl. No.: 13/339,113

(22) Filed: Dec. 28, 2011

Related U.S. Application Data
(63) Continuation-in-part of application No. 12/863,371, filed on Jan. 14, 2011, now abandoned.

(51) Int. Cl. A61K 31/737; A61P 29/00; A61K 9/127

(52) U.S. Cl. 424/450; 514/54

(57) ABSTRACT

The present invention relates to agents for intra-articular injection that contain a mixture of alpha-tocopherol, phospholipids or poloxamers, proteoglycans, and a cortisone crystal suspension or a cortisone crystal solution. The agents are suitable for therapy of rheumatic diseases, in particular of arthrosis.

### CROSS-REFERENCE TO RELATED APPLICATIONS

This application is a Continuation-in-Part of U.S. application Ser. No. 12/863,371, filed Jan. 14, 2011, which is the National Stage of International Application No. PCT/EP2008/000365, filed Jan. 18, 2008, the entire contents of all of which are incorporated herein by reference in their entirety.

### SUMMARY OF THE INVENTION

The present invention relates to medicaments containing vitamin E for intra-articular injection.

The efficacy of vitamin E in the various forms of rheumatic diseases can be considered to be assured. Relatively high doses of 400 mg-1,000 mg are administered for this purpose via the enteral or parenteral route. The therapeutic value of vitamin E being at least partly water-soluble has been recognized and the water solubility of said vitamin was improved by acetate formation. However, the water solubility is thus improved only gradually, rather in the form of a suspension, such that, for example, the acetate form of alpha-tocopherol must only be administered intramuscularly. Intravenous application is therefore precluded.

Intra-articular application would be an interesting therapeutic option for the application of vitamin E, for example in cases of osteoarthritis. However, in this context vitamin E is associated with a disadvantage in that the communication of the articular cavity with the vascular system gives rise to a risk of fat embolism which is the reason that intra-articular injection is expressly contraindicated for pertinent preparations.

The disadvantages illustrated above are remedied by the innovation to be proposed herein. To this end alpha-tocopherol, also the acetate form thereof, is mixed with a phospholipid or a liquid poloxamers. The composition of the phospholipids, ceramides, encephalins or lecithin can vary, but it is preferable to use phosphatidylcholine. In this context, the ideal mixing ratio of alpha-tocopherol to phospholipid or poloxamer is from 1:1 to 1:2.5, preferably 1:2.

When it is introduced into joints, the alpha-tocopherol-phospholipid or alpha-tocopherol-poloxamer mixture is advantageous due to the improved viscosity such that the miscibility with local anesthetics is much improved and the contamination of the entire joint space is ensured due to the improved viscosity. Phospholipids, specifically phosphatidylcholine, have been proven to permeate is well into tissues such that the “carry-along” effect of this substance allows the alpha-tocopherol to exert its membrane-stabilizing or anti-inflammatory effect in the area to be treated more rapidly and better. The same effect is found in poloxamers. Poloxamers are copolymers of ethylene oxide and propylene oxide. Typically, they are composed of a central hydrophobic chain of poly(propylene oxide) flanked by two hydrophilic chains of poly(ethylene oxide). They are commercially available from BASF SE under the tradename Pluronic, also as pharmaceutical grades.

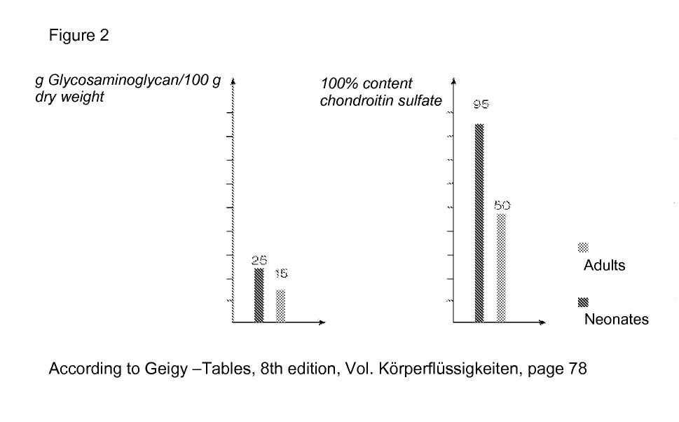

The efficacy of proteoglycans is proven, especially in the context of intra-articular injection. Multiple preparations made of hyaluronic acid are available for this purpose in the pharmaceutical market. Strangely, chondroitin sulfate, a proteoglycan that stands out amongst the members of the group of articular proteoglycans since it is present in elevated levels in the articular cartilage in early youth, see FIG. 2, has thus far not been used for intra-articular injection after pharmaceutically appropriate preparation.

According to the invention, a mixture of 50 mg tocopherol acetate, 150 mg phosphatidylcholine or poloxamer, and 100 mg chondroitin sulfate, which was obtained from shark cartilage and prepared in accordance with pharmaco-legal and pharmaceutical aspects, leads to a clear improvement of the symptoms associated with osteoarthritis in the vast majority of cases, since the healing effects of the individual substances are obviously potentiated in the combination.

Surprisingly, it has been found that the mixture described above is homogenized even further by admixture of folic acid—e.g., 10 mg of folic acid in aqueous solution in the present case. The solutions remains absolutely clear even after months of storage in the cold.

Moreover, said solution proves to be capable of taking up aqueous diclofenac solution. Usually, diclofenac is not applied by the intraarticular route, while, in the mixture described above, it can be applied not only without any hazard, but it also leads to a clear improvement in the tolerability of the applied mixture due to its antiphlogistic effect.

In active arthrotic diseases, it is useful to mix the medication combination described above with cortisone preparations. Surprisingly, it was found that crystal suspensions of dexamethasone acetate that were admixed to the combination described above resulted in symptom relief for up to two years. The application of pure crystal suspensions into the diseased joint is controversial since the crystals are thought to cause additional mechanical wear and tear at the surface of the cartilage. Indeed, the pain-relieving and anti-inflammatory effects of injections of said type usually persist for just a few days or weeks.

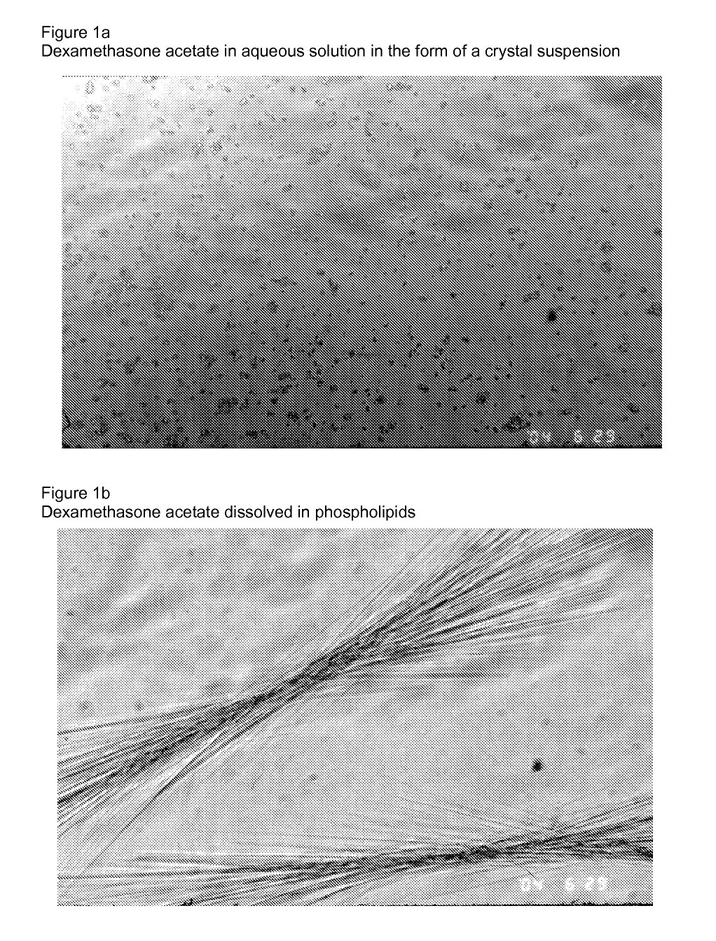

Since the clinical results obtained with the combination described above were very different, the dexamethasone crystals in pure saline and in the above-described innovation were examined by microscopy. According to these studies, the crystals in phospholipid solution were reduced by 50% within a period of six hours and no longer detectable after twelve hours. In contrast, very thin, needle-like formations with structures were seen which obviously are not capable of causing mechanical damage to the cartilage (see FIGS. 1a and 1b). The above-described combination of medications is therefore capable of achieving a previously unknown physiological depot effect by structural conversion of the dexamethasone molecules. The subsequent check-up by HPLC produced proof for the strand-like polymers made of dexamethasone acetate.

The innovation is also advantageous in that fewer injections per joint are required due to the higher efficacy. Moreover, the pain-relieving effect persists for up to 2 years.

There is no more suitable means than oil for reducing the friction on each other exerted by degeneratively changed joint surfaces. However, direct injection of oil into the diseased joint is not free of hazards for the reasons mentioned above (risk of fat embolism).

Basically, various oils, which are already being admixed to medications to achieve a depot effect, would be suitable for this purpose. However, from a physiological point of view, castor oil is optimal in order to reduce the shearing effect. In order to introduce castor oil in a non-hazardous manner, it is therefore proposed herein to form liposomes. The proposed phospholipid or poloxamer is excellently suited for the formation of castor oil liposomes. The formation of liposomes based on phospholipids is part of the prior art. In contrast, the application of liposomes for intraarticular injection, in particular with the components specified above, is not known.

Poloxamers have an even better emulsifying effect than phospholipids, it is possible to emulsify up to 0.2% by weigth of oil in the mixture without liposomes being visible in a microscope. The increased oil amount serves to improve the lubricating effect of the mixture, the reduced mechanical load on the articular surfaces allows a regeneration of the joint. The use of bigger amounts of oil is possible, the liposomes are well tolerated by the joint.

Although castor oil is a highly viscous oil, it can be applied through the finest cannulas that are commercially available when used in the form of the preparation described in the innovation. This renders its application even in the smallest joints, for example digital joints, feasible without any problems.

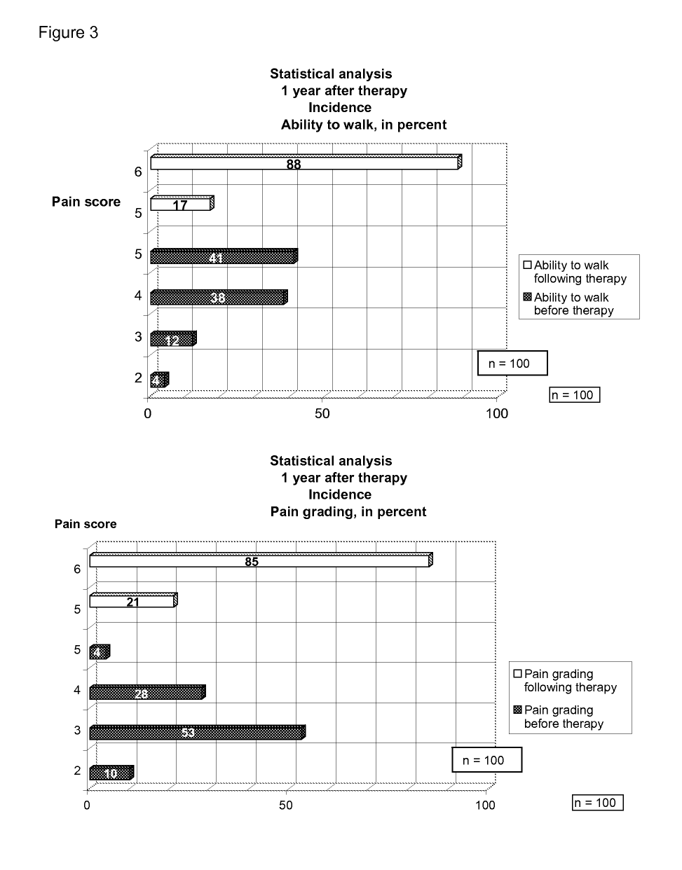

The results of a treatment of said type involving 1-2 injections per joint are shown in the statistical analysis in FIG. 3 which is based on 100 patients predominantly afflicted by osteoarthritis of the knee and hip. The symptom-free interval is comparatively long, approx. 12 months in the standard case.

A further improvement can be achieved by incorporating a 1% procaine in saline solution. The addition lowers the viscosity of the mixture so much that it can be administered with still finer cannulas, e.g such with a size of 17/42. This does not only ease the handling it also minimizes the risks associated with the injection. A smaller puncture means less risk for an infection.

The mixture containing alpha-tocopherol, poloxamer, proteoglycans, and a cortisone crystal suspension or a cortisone crystal solution are so well tolerated that it is not necessary to immobilize the joint after the injection. In case of a knee joint the patient can walk, excessive loads e.g. athletics should be avoided, however.

BRIEF DESCRIPTION OF THE EMBODIMENTS

FIG. 1a is an image of dexamethasone acetate in aqueous solution in the form of crystal suspension.

FIG. 1b is an image dexamethasone acetate dissolved in phospholipids.

FIG. 2 shows data according to Geigy—Tables, 8th edition, Vol. Koerperfluessigkeiten, page 78.

FIG. 3 depicts statistical analytical data 1 year after therapy.

### DETAILED DESCRIPTION OF PREFERRED EMBODIMENTS

1. Agent for intra-articular injection comprising a mixture of alpha-tocopherol, phospholipids or poloxamers, proteoglycans, and a cortisone crystal suspension or a cortisone crystal solution.

2. Agent as in 1. containing chondroitin sulfate as proteoglycan.

3. Agent as in 1. or 2. wherein the mixing ratio of alpha-tocopherol to phospholipids is from 1:1 to 1:2.5.

4. Agent as in any one of 1.-3. wherein the phospholipid is phosphatidylcholine.

5. Agent as in any one of 1.-4. wherein the cortisone crystal solution consists of dexamethasone acetate in phospholipids or poloxamers.

6. Agent as in any one of 1.-5. containing 25 to 75 mg diclofenac in aqueous solution.

7. Agent as in any one of 1.-6. containing 5 to 15 mg folic acid.

8. Agent as in any one of 1.-7. containing liposomes that are formed by adding a medicinal agent-compatible oil to the phospholipid or poloxamer.

9. Agent as in 8. wherein the oil is castor oil.

1. Agent for intra-articular injection, wherein it contains a mixture of alpha-tocopherol, phospholipidsor poloxamers, proteoglycans, and a cortisone crystal suspension or a cortisone crystal solution.

2. Agent according to claim 1, wherein it contains a poloxamer.

3. Agent according to claim 1, wherein it contains a phospholipid and the phospholipid is phosphatidylcholine.

4. Agent according to claim 1, wherein it contains chondroitin sulfate as proteoglycan.

5. Agent according to claim 2, wherein it contains chondroitin sulfate as proteoglycan.

6. Agent according to claim 1, wherein the mixing ratio of alpha-tocopherol to phospholipids or poloxamers is from 1:1 to 1:2.5.

7. Agent according to claim 1, wherein the cortisone crystal solution consists of dexamethasone acetate in phospholipids or poloxamers.

8. Agent according to claim 1, wherein it contains 25 to 75 mg diclofenac in aqueous solution.

9. Agent according to claim 2, wherein it contains 25 to 75 mg diclofenac in aqueous solution.

10. Agent according to claim 1, wherein it contains 5 to 15 mg folic acid.

11. Agent according to claim 2, wherein it contains 5 to 15 mg folic acid.

12. Agent according to claim 1, wherein it contains liposomes that are formed by adding a medicinal agent-compatible oil to the phospholipid or poloxamer.

13. Agent according to claim 12, wherein the oil is castor oil.

14. Agent according to claim 1, wherein a solution of 1% procaine in saline is contained.

15. Agent according to claim 2, wherein a solution of 1% procaine in saline is contained.

16. Method of treatment of forms of rheumatic disease, in particular arthrosis, and even more particularly osteoarthritis wherein an agent according to claim 1 is injected into a diseased joint.

17. Method of treatment of forms of rheumatic disease, in particular arthrosis, and even more particularly osteoarthritis wherein an agent according to claim 2 is injected into a diseased joint.

18. Method of treatment of forms of rheumatic disease, in particular arthrosis, and even more particularly osteoarthritis wherein an agent according to claim 8 is injected into a diseased joint.

19. Method of treatment of forms of rheumatic disease, in particular arthrosis, and even more particularly osteoarthritis wherein an agent according to claim 12 is injected into a diseased joint.

20. Method of treatment of forms of rheumatic disease, in particular arthrosis, and even more particularly osteoarthritis wherein an agent according to claim 15 is injected into a diseased joint.
